(19) United States
(12) Patent Application Publication
(10) Pub. No.: US 2026/0271887 A1
(43) Pub. Date: Sep. 17, 2026

(54) SYSTEM AND METHOD FOR MONITORING AND COMMUNICATING WITH CONFINED ANIMALS

(71) Applicant: Lacie Leatherman, Mulvane, KS (US)

(72) Inventor: Lacie Leatherman, Mulvane, KS (US)

(21) Appl. No.: 19/672,090

(22) Filed: May 8, 2026

Related U.S. Application Data
(63) Continuation of application No. PCT/US2024/054985, filed on Nov. 7, 2024.
(60) Provisional application No. 63/597,028, filed on Nov. 8, 2023.

(51) Int. Cl. A01K 29/00; A01K 1/035; A61B 5/0205; A61B 5/024; A61B 5/087; A61B 5/145

(52) U.S. Cl. CPC A01K 29/00; A01K 1/035; A61B 5/02055; A61B 5/024; A61B 5/087; A61B 5/14532; A61B 5/14542; A61B 2503/40

(57) ABSTRACT

Systems and methods for an animal monitoring and communication. The animal monitoring and communication system includes a first sensor configured to be positioned proximal to an environment of an animal, an environment device configured to modify the environment of the animal, and a processor communicatively coupled to the first sensor and to the environment device. The processor is configured to receive a first condition from the first sensor, compare the first condition to a first threshold condition, and initiate an action within the environment of the animal in response to the first condition reaching the first threshold condition.

### CROSS-REFERENCE TO RELATED APPLICATION

This application is a continuation of International Patent Application No. PCT/US2024/054985, filed on Nov. 7, 2024, entitled “SYSTEM AND METHOD FOR MONITORING AND COMMUNICATING WITH CONFINED ANIMALS,” which in turn claims priority to U.S. Provisional Patent Application No. 63/597,028, filed Nov. 8, 2023, entitled “SYSTEM AND METHOD FOR MONITORING AND COMMUNICATING WITH CONFINED ANIMALS,” which are incorporated by reference herein, in their entirety and for all purposes.

### BACKGROUND

In the field of animal wellness and transport, such as during rehoming, vet visits, K9 units, vacations, or when a caregiver is away from an animal, the animal's experience of isolation and/or transport can result in uneasy experiences for the animal and its caregiver. Although some solutions are available, such as playing music to soothe the animal, improvements to animal experience, safety, and overall health are needed.

### SUMMARY

According to certain implementations, an animal monitoring and communication system includes a first sensor configured to be positioned proximal to an environment of an animal, an environment device configured to modify the environment of the animal, and a processor communicatively coupled to the first sensor and to the environment device. The processor is configured to receive a first condition from the first sensor, compare the first condition to a first threshold condition, and initiate an action within the environment of the animal in response to the first condition reaching the first threshold condition.

In implementations, the first sensor includes one or more of a camera, thermometer, a humidity sensor, a light sensor, or a combination thereof. The initiated action may cause the processor to receive monitoring data from the first sensor. The processor may be communicatively coupled to a communications network and configured to transmit monitoring information to a communications hub. The processor may be communicatively coupled to a user interface and configured to transmit monitoring information thereto. The processor may be configured to receive input from the user interface for controlling the environment device.

In some implementations, the animal monitoring and communication system may include a user device communicatively coupled to the processor, the user device configured to display the user interface. The user device may be configured to be docked in a docking station of the animal monitoring and communication system. The processor may be configured to transmit monitoring information to the user device upon being docked in the docking station. The docking station may include a housing configured to be attached to a housing of the environment of the animal. The housing of the environment of the animal may be an animal crate or an animal stall. In some implementations, the processor, the first sensor, and the environment device may be integrated into a single assembly. In some implementations, the processor, the first sensor, and the environment device may be arranged in the environment of the animal and remotely located from each other.

According to alternative or additional implementations, the animal monitoring and communication system includes a second sensor configured to monitor a second condition. The second sensor and the processor may be arranged in the environment of the animal and remotely located from each other. The second sensor may include one or more of a clothing article, a collar, a saddle blanket, a mat, an implantable chip, or a combination thereof. The second condition may be one or more of an animal temperature, a heart rate, a respiration rate, a blood glucose level, an oxygen level, or a combination thereof. The initiated action may cause the processor to receive monitoring data from the second sensor. The first threshold condition may be predefined by a user. The first threshold condition may be generated by one or more of a machine learning model, artificial intelligence, or a combination thereof. The environment device may include one or more of a fan, a light, a speaker, a feeding device, an electric device, or a combination thereof. The initiated action may cause the environment device to modify the environment of the animal.

According to alternative or additional implementations, a system includes one or more hardware processors and a memory storing instructions that, when executed by at least one of the one or more hardware processors, causes the system to perform operations. The operations include receiving a first condition from a first sensor proximal to an environment of an animal, comparing the first condition to a first threshold condition, and initiating a first action within the environment of the animal in response to the first condition reaching the first threshold condition. The system may include receiving a second condition from a second sensor, comparing the second condition to a second threshold condition, and initiating a second action within the environment of the animal in response to the second condition reaching the second threshold condition.

According to alternative or additional implementations, a non-transitory computer-readable storage medium includes instructions that, when executed by one or more hardware processors of a system, cause the system to perform operations. The operations include receiving a first condition from a first sensor proximal to an environment of an animal, comparing the first condition to a first threshold condition, and initiating a first action within the environment of the animal in response to the first condition reaching the first threshold condition.

### BRIEF DESCRIPTION OF THE DRAWINGS

FIGS. 1A and 1B illustrate an assembly of an animal monitoring and communication system according to implementations of the present disclosure.

FIG. 2A illustrates a housing of the animal monitoring and communication system according to implementations of the present disclosure.

FIG. 2B illustrates an animal monitoring and communication device of the animal monitoring and communication system according to implementations of the present disclosure.

FIGS. 3A and 3B illustrate a docking station of the animal monitoring and communication system according to implementations of the present disclosure.

FIG. 4 illustrates a user interface of a user device communicatively coupled to the animal monitoring and communication system according to implementations of the present disclosure.

FIGS. 5A-5C illustrate schematic block diagrams of a communication system for the animal monitoring and communication system according to implementations of the present disclosure.

FIG. 6 is a flow chart of a method for initiating an action based on a condition with the animal monitoring and communication system according to implementations of the present disclosure.

FIG. 7 is a flow chart of a method for receiving monitoring data with the animal monitoring and communication system according to implementations of the present disclosure.

FIG. 8 is a flow chart of a method for initiating an action based on an action indicator with the animal monitoring and communication system according to implementations of the present disclosure.

FIG. 9 is a flow chart of a method for initiating an action based on a received threshold value with the animal monitoring and communication system according to implementations of the present disclosure.

FIG. 10 is a flow chart of a method for initiating an action based on two conditions reaching a threshold with the animal monitoring and communication system according to implementations of the present disclosure.

FIG. 11 is a flow chart of a method for monitoring a recovery of an animal or an environment return with the animal monitoring and communication system according to implementations of the present disclosure.

### DETAILED DESCRIPTION

In the following description, for the purposes of explanation, numerous specific details are set forth in order to provide an understanding of various example embodiments of the present subject matter. It will be evident, however, to those skilled in the art, that example embodiments of the present subject matter may be practiced without these specific details.

The present disclosure relates to providing a compact, portable animal monitoring and communication system for use in connection with animal enclosures, such as pet crates, animal stalls, vehicles, and so on. In some implementations, the animal monitoring and communication device may be configured to be removably positioned and/or attachable to animal enclosures or to fixed devices proximate the animal enclosure, such as tripod, a crate, a door, ceiling or wall of an enclosure. In other implementations, the animal monitoring and communication device may be integrated with the animal enclosure.

The animals that may benefit from the animal monitoring and communication system may include an animal in confinement, which may include but is not limited to companion animals, dogs, cats, rabbits, horses, ponies, pigs, birds, cattle, and so on.

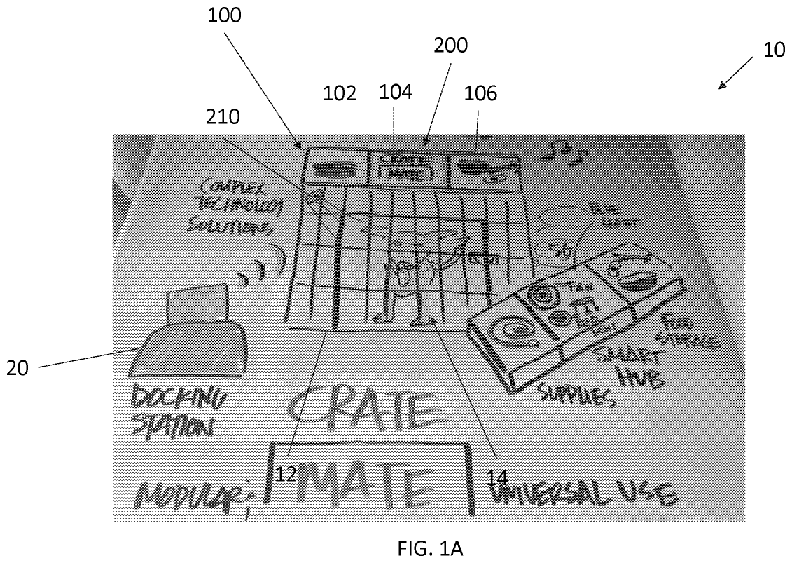

Referring to FIGS. 1A and 1B, an assembly of an animal monitoring and communication system 10 may include a docking station 20, a housing 100, and an animal monitoring and communication device 200 configured for monitoring the animal and taking action, for in response to the monitoring. The animal monitoring and communication system 10 or components thereof may be placed in proximity to an animal enclosure 12. The animal enclosure 12 may enclose, either fully, partially, or enspace, a target animal 14. Because the target animal 14 may include companion animals, dogs, cats, horses, ponies, pigs, birds, cattle, and so on, the animal enclosure may be a crate, a kennel, a stall, a pen, a cage, a vehicle (e.g., a motor vehicle), and so on.

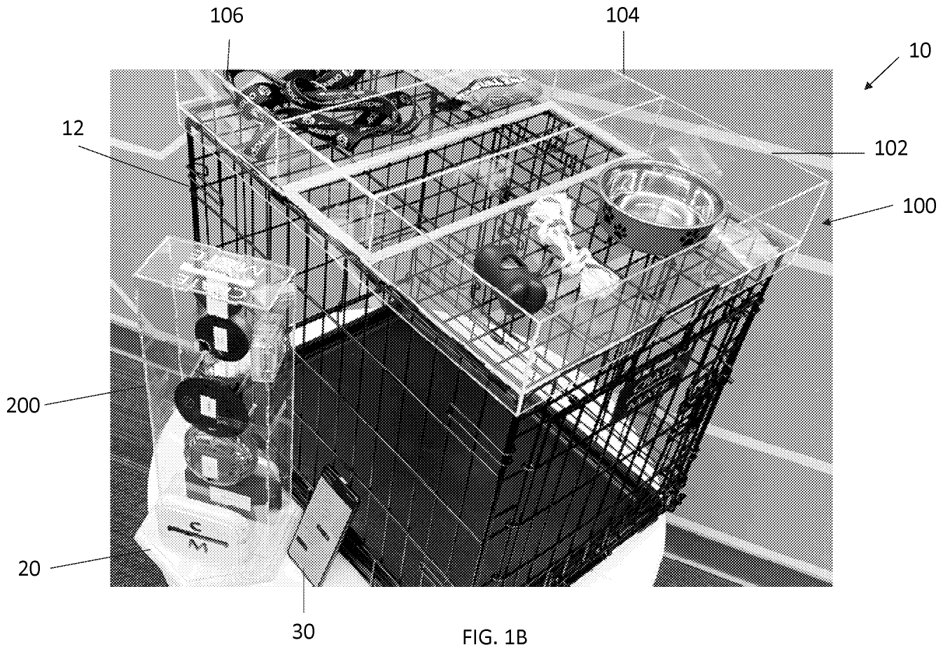

The housing 100 may be configured to sit atop the animal enclosure 12, attach to the animal enclosure 12, hang from the animal enclosure 12, or sit proximal the animal enclosure 12. The housing 100 may be configured to receive various components of the animal monitoring and communication system 10, such as one or more components of the animal monitoring and communication device 200. The housing 100 is discussed in further detail with respect to FIG. 2A.

The animal monitoring and communication device 200 may include a processing unit 202, a sensor 204, an environment device 206, and/or a combined sensor and environment device 208. The animal monitoring and communication device 200 may be configured to be positioned proximal the animal enclosure 12 and/or the target animal 14, and for instance, may sit atop the animal enclosure 12, attach to the animal enclosure 12, hang from the animal enclosure 12, sit proximal the animal enclosure 12, be received by the housing 100, and/or be docked via the docking station 20.

The sensor 204 may be configured to sense environment conditions, animal conditions, or a combination thereof. Sensed environment conditions may include but are not limited to: temperature, pressure, humidity, sound level, light level, movement, or a combination thereof. Sensed animal conditions may include but are not limited to: animal temperature, heart rate, respiration rate, blood glucose level, oxygen level, or a combination thereof.

The environment device 206 may be configured to cause changes to the environment of the animal enclosure 12. For example, the environment device 206 may be configured to adjust an environmental parameter or condition. Environmental parameters or conditions may include but are not limited to: a temperature, light level, sound level, humidity, and so on of the environment of the enclosure 12. The environment device 206 may adjust the environment parameter or condition by switching on/off, increasing or decreasing an intensity, or a combination thereof.

The combined sensor and environment device 208 may be configured to sense conditions, caused changes in the environment of the animal enclosure 12, or a combination thereof. The combined sensor and environment device 208 may be configured to sense environment conditions, animal conditions, or a combination thereof. Sensed environment conditions may include but are not limited to: temperature, pressure, humidity, sound level, light level, movement, or a combination thereof. Sensed animal conditions may include but are not limited to: animal temperature, heart rate, respiration rate, blood glucose level, oxygen level, or a combination thereof. To cause changes in the environment, the combined sensor and environment device 208 may be configured to adjust an environmental parameter or condition. Environmental parameters or conditions may include but are not limited to: a temperature, light level, sound level, humidity, and so on of the environment of the enclosure 12. The combined sensor and environment device 208 may adjust the environment parameter or condition by switching on/off, increasing or decreasing an intensity, or a combination thereof.

The animal monitoring and communication system 10 may include one or more external devices 210. The external devices 210 may be communicatively coupled to the animal monitoring and communication device 200, the docking station 20, the user device 30, or a combination thereof. For instance, the external device 210 may include at least one processor and memory and be configured to be communicatively coupled to the animal monitoring and communication system 10 components and transmit monitoring information to thereto.

For instance, the external devices 210 may include one or more sensors configured for monitoring the animal, such as the animal's temperature, heart rate, respiration rate, blood glucose, oxygen level, and so on. The external devices 210 may be configured to be positioned proximal the enclosure 12 of the target animal 14. The external device 210 may be positioned at a distance from the animal monitoring and communication device 200. The external devices 210 may be a sensor, environment device, or a combination thereof. For example, the external devices 210 may be a wearable device, such as an article of clothing, a collar, a saddle blanket, an implantable chip, an adhesive patch, and so on. The external devices 210 may be a mat configured to be on the floor or ceiling of the animal enclosure 12. The mat may include one or more sensors (e.g., a network of sensors) to monitor animal conditions.

The external devices 210 may additionally or alternatively be configured to sense environment conditions of the animal enclosure 12 and transmit the sensed information to the animal monitoring and communication device 200 (e.g., to the processing unit 202). The sensed information may include temperature, pressure, humidity, pressure, sound level, light level, movement, or a combination thereof.

The external devices 210 may additionally or alternatively be configured to adjust an environmental parameter. For example, the external device 210 may include a fan, a speaker, a light (e.g., a red light, a blue light, and so on), an ozone machine (e.g., to kill bacteria). The animal monitoring and communication device 200 is discussed in further detail with respect to FIG. 2B.

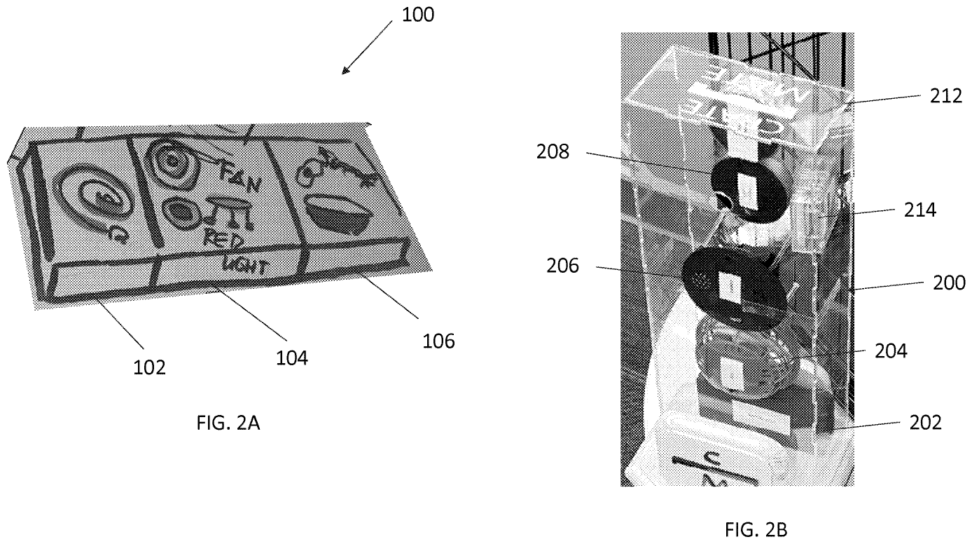

As described in further detail below, the docking station 20 may be configured to dock, e.g., receive, the animal monitoring and communication device 200 and/or the user device 30. The docking station 20 may be configured to sit atop the animal enclosure 12, attach to the animal enclosure 12, hang from the animal enclosure 12, or sit proximal the animal enclosure 12. The docking station 20 may be configured to be communicatively coupled to the animal monitoring and communication device 200, the user device 30, the external sensors 210, or a combination thereof. The docking station is discussed in further detail with respect to FIGS. 3A and 3B.

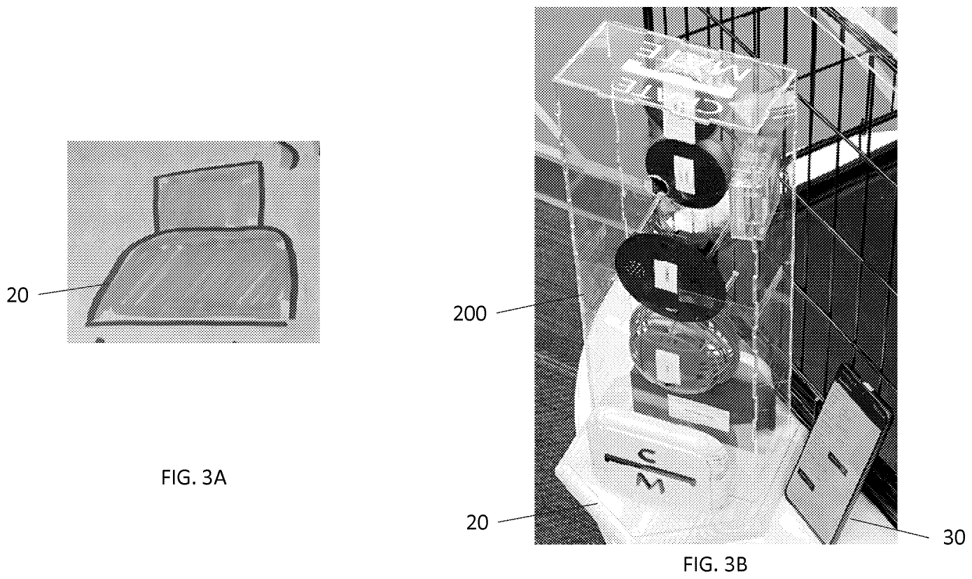

The user device 30 may be a handheld device (e.g., a smart phone, smart watch, tablet, laptop, and so on). The user device 30 may be communicatively coupled to the animal monitoring and communication device 200, the docking station 20, the external sensor 210, or a combination thereof. The user device 30 may be portable. The user device 30 may include a user interface 31 configured to receive monitoring information. The user interface 31 may provide control of environment device 206, combined sensor and environment device 208, external device 210, or a combination thereof. The user device 30 is discussed in further detail with respect to FIG. 4.

Referring to FIG. 2A, the housing 100 of the animal monitoring and communication system 10 may include multiple sections, e.g., three sections. For example, the housing 100 may include a first section 102 for receiving/storing supplies, a second section 104 for receiving the animal monitoring and communication device 200, and a third section 106 for storing/receiving supplies. The first section 102 and/or third section 106 may store supplies for the target animal 14, such as a leash, a collar, a toy, food, treats, and so on. It will be appreciated that the housing may include more or less than three sections, for instance, based on the target animal 14, and/or the size, and/or configuration of the animal enclosure 12.

Referring to FIG. 2B, the animal monitoring and communication device 200 of the animal monitoring and communication system 10 may include a housing 212. The housing 212 may be configured to be removable as a single unit from the housing 100 (e.g., second section 104) of FIG. 2A. The housing 212 may provide protection for electrical components of the animal monitoring and communication device 200. For example, the housing 212 may enclose the processing unit 202, the sensor 204, the environment device 206, and/or the combined sensor and environment device 208. In some instances, the housing 212 may include a hinge 214, latch, or other mechanism for opening and closing the housing 212. The housing 212 may open to provide access to an interior of the housing 212. Accordingly, the housing 212 and components therein may be configurable. In some instances, the sensor 204, the environment device 206, and/or the combined sensor and environment device 206 may be interchangeable or removable, such that a different configuration or combination of sensors and environment devices may be housed in the housing 212 of the animal monitoring and communication device 200. In some implementations, the housing 212 includes electrical receptacles. In some implementations, the portion of the second section 104 configured for holding the animal monitoring and communication device 200 may include specially configured electrical receptacles for receiving electrical components designed to have complementary electrical connectors to such receptacles.

In some implementations, one or more of the other electrical components may be fixed within the housing 212, while in other implementations, one or more of the electrical component modules may be removably attachable to the housing 212. For instance, the external device 210 may be arranged in the assembly during transport, but may be removed from the assembly and joined to the animal enclosure 12 during use. In such cases, the external device 210 may communicatively coupled to the communication module via a wired or wireless connection thereto. In such cases, the external device 210 may be powered by the animal monitoring and communication device 200 or from a separate power source. The housing 212 of the animal monitoring and communication device 200 may also include batteries, timers, and other electronic devices to facilitate monitoring of the animal environment and modification of the environment.

The electrical receptacles of the housing 212 may be configured to receive a variety of electrical components such as communication modules configured to communicate via a wireless or wired connection to the processing unit 202, sensor 204, environment device 206, and/or combined sensor and environment device 208. The animal monitoring and communication device 200 may be configured to be powered by an external power source. The animal monitoring and communication device 200 may include a stored power source, such as a battery. The animal monitoring and communication device 200 may be configured to provide power to the sensor 204, environment device 206, and/or combined sensor and environment device 208. The animal monitoring and communication device 200 may be configured to transmit and receive data, e.g., via the processing unit 202.

The sensor 204 may be communicatively coupled to the various components of the animal monitoring and communication system 10. The sensor 204 may include multiple sensors. The sensor 204 may provide real time information about the target animal 12 and/or the animal's environment to communicatively coupled devices of the animal monitoring and communication system 10. In some instances, the sensor 204 may provide recorded or stored information to the processing unit 202, docking station 20, the user device 30, or a combination thereof. The sensor 204 may monitor and/or detect environment conditions, animal conditions, or a combination thereof. The sensor 204 may monitor environment conditions such as ambient temperature, pressure, humidity, pressure, sound level, light level, movement, or a combination thereof. The sensor 204 may monitor animal conditions including animal temperature, heart rate, respiration rate, blood glucose, oxygen level, movement of the target animal 14. In some instances, the sensor 204 may be a motion sensor, video camera, microphone, and so on.

The environment device 206 may be communicatively coupled to the various components of the animal monitoring and communication system 10. The environment device 206 may include multiple environment devices. The environment device 206 may receive real time input from the processing unit 202, the docking station 20, the user device 30, or a combination thereof. The environment device 206 may adjust an environment condition of the environment of the target animal 14 by adjusting a parameter of the environment device 206. For example, the environment device 206 may adjust an environment condition of the environment of the animal enclosure 12. For example, the environment device 206 may adjust temperature, light level, sound level, humidity, and so on, of the environment of the enclosure 12. The environment device 206 may be a speaker, light, therapy light (e.g., infrared lights, blue lights), feeding device, watering device, fan, heater, and so on.

The combined sensor and environment device 208 may be communicatively coupled to the various components of the animal monitoring and communication system 10. The combined sensor and environment device 208 may include multiple devices. The combined sensor and environment device 208 may provide real time information about the target animal 12 and/or the animal's environment (e.g., enclosure 12) to the processing unit 202, docking station 20, the user device 30, or a combination thereof. The combined sensor and environment device 208 may receive real time input from the processing unit 202, the docking station 20, the user device 30, or a combination thereof. The combined sensor and environment device 208 may be a device that is capable of providing both monitoring and environment modification. In some instances, the combined sensor and environment device 208 may be a device that includes a camera, speaker, and microphone.

Referring to FIGS. 3A and 3B, the docking station 20 of the animal monitoring and communication system 10 may be configured to receive, e.g., dock, the animal monitoring and communication device 200 the user device 30, or both. For example, the animal monitoring and communication device 200, the user device 30, or both, may be docked in the docking station 20.

The docking station may be configured to charge the animal monitoring and communication device 200 and/or the user device 30 when docked. When docked, the animal monitoring and communication device 200 and/or the user device 30 may continue to monitor the target animal 14 and the environment (e.g., enclosure 12). The animal monitoring and communication device 200 may be configured to be un-docked for portability. Upon docking, the processor of the animal monitoring and communication device 200 may be programmed to transmit information, for instance to the user device 30 and/or the network. In some cases, the animal monitoring and communication device 200 may continuously upload data to the user device 30 and/or the network regardless of docking position.

Referring to FIG. 4, the user device 30 may include a user interface 31. The user device 30 may be communicatively coupled to the animal monitoring and communication device 200 and the docking station 20. The user interface 31 may be configured to display monitoring data/information and transmit instructions to the animal monitoring and communication device 200 via the user device 30. In some implementations, the animal monitoring and communication device 200 may be configured to be remotely controlled by the user. The user interface 31 may receive and display environment data 32, location data 34, environment modification 36, environment modification timing 38.

The environment data 32 displayed on the user interface 31 may relate to the environment of the animal enclosure 12. For example, the environment data 32 may include a temperature, pressure, ambient light, sound level, and/or statuses of the devices of the animal monitoring and communication system 10. The location information 34 displayed on the user interface 31 may include location information for the devices of the animal monitoring and communication system 10 or enclosure 12. The environmental modification 36 displayed on the user interface 31 may be configured to enable a user to adjust a parameter of environmental devices (e.g., environment device 306) of the animal monitoring and communication device 200, such as fans, heaters, lights, speakers, and so on. The environmental modification 36 may turn the environmental devices 306 on and off. The environment modification timing 38 displayed on the user interface 31 may enable a user to set the environmental devices of the animal monitoring and communication device 200 to an on or off status or an intensity of the device for a specified period of time.

More generally, the user may control the various electrical components of the animal monitoring and communication device 200, such as by speaking into a microphone communicatively coupled to a speaker of the animal monitoring and communication device 200, by instructing that music be played by the speaker, by adjusting a light status (e.g., on/off) or intensity, temperature, and so on.

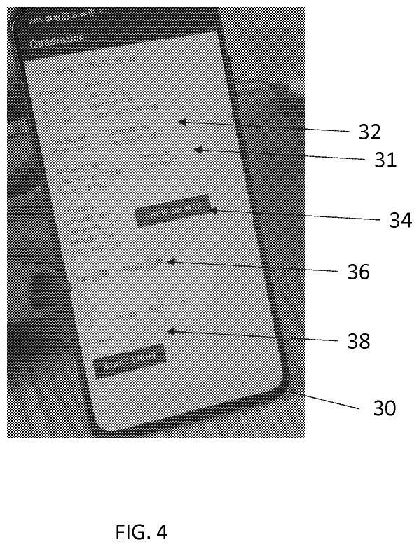

Referring to FIGS. 5A-5C, a schematic block diagram is provided for a networking environment for the animal monitoring and communication system 10. The networking environment may include a network 300, user device(s) 302, sensor(s) 304, environment devices(s) 306, animal monitoring and communication device(s) 310, and communications hub 316.

The network 300 may comprise one or more networks, such as local area networks (LANs), wide area networks (WANs), metropolitan area networks (MANs), cellular networks, and/or the Internet. Communications provided to, from, and within the network 300 may be wired and/or wireless, and further may be provided by any networking devices known in the art. Devices communicating over the network 300 may communication with any communications protocol, including TCP/IP and UDP protocols. The network 300 may comprise long range radio technology to connect to the WANs using low power. The network 300 may enable internet of things communication. Moreover, user devices 302 may be configured to communication using known protocols such as HTTP, HTTPS, SSL, or any protocol derived therefrom.

User device(s) 302 may be a handheld user device. For example, the user device 302 may be a smartphone, smart watch, tablet, laptop, another computing device, and so on. The user device 302 may be communicatively coupled to the network 300 and the animal monitoring and communication device 200. In another instance, the user device 302 may be communicatively coupled to the network 300 and the communications hub 316. In another instance, the user device 302 may be communicatively coupled to the network 300. The user device 302 may be configured to receive monitoring data from the communicatively coupled devices. The user device 302 may display received monitoring data via a user interface. In some instances, the user device 302 may include one or more third party applications that include a user interface. The user interface may receive monitoring data from the animal monitoring and communication device 310. The user device 302 may be configured to transmit an action indicator or action indicators to the communicatively coupled devices, including the environment device 306 and animal monitoring and communication device 310.

Sensor(s) 304 may be any of the sensors described with respect to FIGS. 1A-2B. The sensor 304 may be communicatively coupled to the animal monitoring and communication device 200. In another instance, the sensor 304 may be communicatively coupled to the communications hub 316. In another instance, the sensor 304 may be communicatively coupled to the network 300. The sensor 204 may be configured to transmit monitoring data to the communicatively coupled devices, record data, and store data.

Environment device(s) 306 may be any of the environment devices described with respect to FIGS. 1A-2B. The environment device 306 may be communicatively coupled to the animal monitoring and communication device 200. In another instance, the environment device 306 may be communicatively coupled to the communications hub 316. In another instance, the environment device 306 may be communicatively coupled to the network 300. The environment device 306 may be configured to receive a parameter modification from the communicatively coupled devices, including user device 302 and animal monitoring and communication device 310.

The animal monitoring and communications device 310 may include a processing unit 312, a computer readable media 314, and a storage 318. In some implementations, as shown in FIG. 5C, the animal monitoring and communications device 310 may include a communications hub 316. Herein, the term computer readable media is used to refer to a single computer readable media in some instances, and in other instances, multiple computer readable in communication with one or more processing units 312. The computer readable media 314 may store executable instructions for the animal monitoring and communication device 310. In another instance, the storage 318 may store executable instructions for the animal monitoring and communication device 310. Computer readable media 314 and/or storage 318 may include any form of computer readable storage or computer readable memory, transitory or non-transitory, including but not limited to, externally or internally attached hard disk drives, solid-state storage, tiered storage solutions, storage area networks, network attached storage, and/or optical storage. The instructions stored on the computer readable media 314 and/or storage 318 may be executed on the one more processing units 312. The processing unit 312 may be configured to transmit monitoring data/information to other components, such as the user device 302, a user interface of the user device 302, the communications hub 316, the environment device 306, and/or network 300. The processing unit 312 may be configured to receive input from the user device 302, or a user interface thereof. The input received from the user device 302 may include parameter modification to control the environment device 306. The processing unit 312 may be configured to modify the parameter of the environment device 306 based on the received input.

The communications hub 316 may be the docking station 20 of FIGS. 1A-3B. The communications hub 316 may be a router. The communications hub 316 may provide a wired connection, such as a USBC connection, or wireless connection such as cellular communications, Bluetooth communications, WiFi communications, satellite communications and combinations thereof. In some implementations, the communication hub 316 may be fixed to the animal monitoring and communication device 310, while in other implementations, the communications hub 316 may be removably attached to the animal monitoring and communication device 310. The other electrical components, such as the user device 302, the sensors 304, and/or the environment device 306 may be communicatively coupled to the communications hub 316. For instance, the electrical components may transmit information, such as monitoring data, to the communications hub 316 and the communications hub 316 may transmit monitoring data to the electrical components. The communications hub 316 may transmit action indicators and parameter modifiers to the electrical components, including the animal monitoring and communication device 310.

In some instances, the networking environment may provide a networked system of multiple animal monitoring and communication devices 310, user devices 302, sensors 304, and environment devices 306. The networking environment may provide a networked system of multiple animal monitoring and communication systems, each including the animal monitoring and communication device 310, the sensor 304, and the environment device 306, to one or more user devices 302. Such a configuration allows for individual control of multiple animal monitoring and communication systems in a similar location, such as neighboring stalls, crates, or enclosures. When multiple user devices 302 are on the networking environment, the user devices 302 may include permissions for individual animal monitoring and communication systems that a given user device may receive monitoring data or transmitting action indicators.

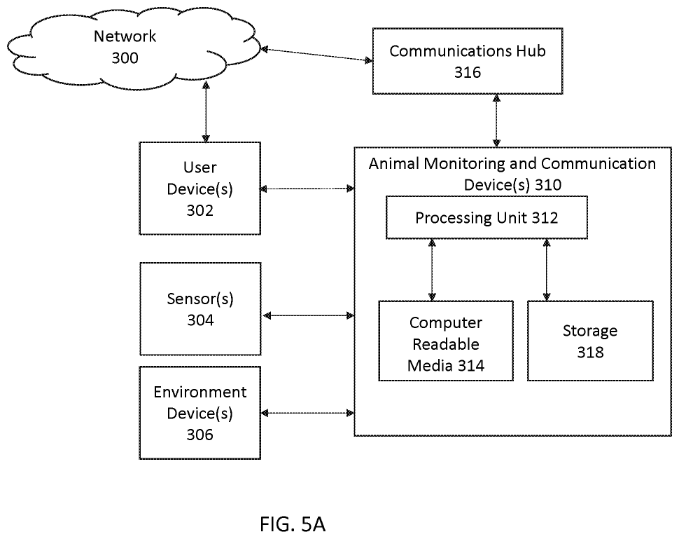

Referring to FIGS. 6-11 generally, in some implementations of use, the animal monitoring and communication device 310 may be configured to activate monitoring and/or alerts upon the one or more sensors 304 detecting an activity or a threshold activity by the animal or environment. Detected activities may include but are not limited to animal movements, types of movements, sounds, types of sounds, animal's temperature, heart rate, respiration rate, blood glucose, oxygen level. In some cases, the threshold may be a predefined threshold of sensed animal behavior, e.g., 10 seconds of movement or barking, and/or environmental conditions while in other cases, the threshold of sensed animal behavior and/or environmental conditions may be adjusted manually by a user, by machine learning models, or by use of artificial intelligence. For instance, the processor may be configured to receive or generate adjusted thresholds based on monitoring information generated by the device and/or based on information received from communicatively coupled devices such as collars, wearable devices, environmental sensors or information generated by other devices communicatively coupled over the network.

Referring to FIG. 6, a method 600 is provided for initiating an action based on a condition with the animal monitoring and communication device 310. The method may be implemented using the communication systems of FIGS. 5A-5C.

At a step 602, the animal monitoring and communication device 310 may receive a first condition. For example, the first condition may be received from sensor(s) 304. The first condition may be an environmental condition of the animal enclosure 12. For example, the first condition may be a temperature, a humidity, a light level, a movement of the target animal 14, a sound, a pressure, or so on. The first condition may be an animal condition of the target animal 14. For example, the first condition may be a temperature, heart rate, respiration rate, blood glucose level, oxygen level, or so on, of the target animal 14. The first condition may be one or more conditions.

At a step 604, the animal monitoring and communication device 310 compares the first condition to a threshold 604. The threshold may be a threshold related to the first condition. The threshold may be a predefined threshold. For instance, the threshold may be a threshold of the environmental condition. The threshold may be a threshold of the animal condition. The threshold may be predefined by a user. In some instances, when the first condition is more than one condition, if a single condition reaches their respective threshold, the threshold of step 604 is reached. In another instance, when the first condition is more than one condition, a designated number of conditions must reach their respective thresholds for the threshold of 604 to be reached. If another instance, when the first condition is more than one condition, all conditions must meet their respective thresholds for the threshold of 604 to be met. If the threshold is not reached, the method 600 returns to step 602, receiving the first condition.

If the threshold is reached, at a step 606, the animal monitoring and communication device 310 initiates an action. The action may include transmitting an alert to the user device 302. The user device 302 may receive an identification of the first condition, and the threshold reached. In addition, or as an alternative to an alert, the action may be taken in the environment of the animal enclosure by adjusting a parameter of the environment device 306. For example, the action may actuate a fan, a heater, a light, cause a sound to be generated, cause an environment device to be turned on and/or off, modify an intensity of an environment device, and so on, via a transmission from the animal monitoring and communication device 310. The action may be an action of a wearable device. For example, the action may cause the wearable device to vibrate, make a sound, and so on. In some cases, the alert may be updated and/or the action may cease upon the animal condition and/or environment condition returning to a baseline level. For example, the environment device may be returned to the state before the action was taken.

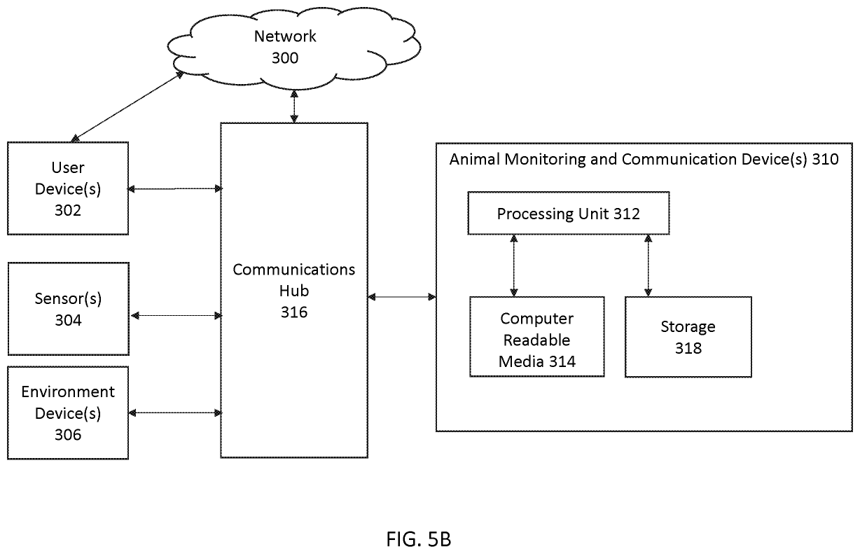

Referring to FIG. 7, a method 700 is provided for receiving monitoring data with the animal monitoring and communication device 310. The method may be implemented using the communication systems of FIGS. 5A-5C.

At a step 702, the animal monitoring and communication device 310 may receive a first condition. Receiving the first condition at step 702 may be substantially similar to receiving a first condition at step 602 of method 600.

At a step 704, the animal monitoring and communication device 310 may compare the first condition to a threshold. Comparing the first condition to a threshold at 704 may be substantially similar to comparing the first condition to a threshold at step 604 of method 600. If the threshold is not reached, the method 700 returns to step 702, receiving the first condition.

If the threshold is reached, at a step 706, the animal monitoring and communication device 310 may receive monitoring data. The monitoring data may be monitoring data of the first condition. The data may be recorded data, recorded data from a predetermined amount of time before the threshold was reached, real time data, or a combination thereof. The monitoring data may be monitoring data relating to a second condition different from the first condition. For example, if the first condition is an environment condition, the second condition may be an animal condition. In another instance, both conditions may be different environment conditions or different animal conditions. The monitoring data may be received such that an artificial intelligence or machine learning program of the animal monitoring and communication device may analyze the data and subsequently modify settings, thresholds, or parameters of the animal monitoring and communication device 310 or track health and well-being. The monitoring data may be received to count a number of occurrences when the first condition has reached the threshold.

At a step 708, the animal monitoring and communication device 310 may optionally transmit monitoring data. The monitoring data may be transmitted to the user device 302. The monitoring data may be transmitted to the user device 302 upon the animal monitoring and communication device 310 being docked in the docking station 20. The monitoring data may be transmitted to the user device 302 such that an action may be designated by the user interface of the user device 302. The user device 302 may be controlled by the caregiver, such as through a handheld device (e.g., smart watch, smartphone, tablet, laptop) or another computing device. For instance, the monitoring information may be transmitted to a user interface of a user device of an owner, veterinarian, a care giver, officer, and so on, of the target animal 14. The monitoring data may be transmitted to an artificial intelligence or machine learning program for analysis of the data and subsequent modification of settings, thresholds, or parameters of the animal monitoring and communication device 310 to track health and well-being. The monitoring data may be transmitted to count a number of occurrences when the first condition has reached the threshold.

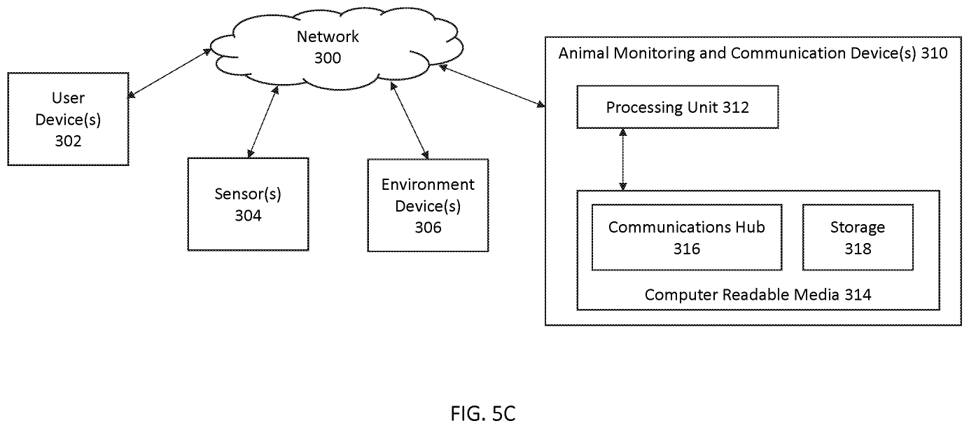

Referring to FIG. 8, a method 800 is provided for initiating an action based on an action indicator with the animal monitoring and communication device 310. The method may be implemented using the communication systems of FIGS. 5A-5C.

At a step 802, the animal monitoring and communication device 310 may receive a first condition. Receiving the first condition at step 802 may be substantially similar to receiving a first condition at step 602 of method 600.

At a step 804, the animal monitoring and communication device 310 may compare the first condition to a threshold. Comparing the first condition to a threshold at 804 may be substantially similar to comparing the first condition to a threshold at step 604 of method 600. If the threshold is not reached, the method 800 returns to step 802, receiving the first condition.

If the threshold is reached, at a step 806, the animal monitoring and communication device 310 may transmit an alert. The alert may be transmitted to the user device 302. The alert may be transmitted to multiple user devices 302. The alert may include monitoring data for the first condition. The alert may indicate that the first condition reached the threshold. The alert may include a set of predetermined environment parameters for modification. For example, if the first condition is a temperature of the environment, the alert may provide the option to turn on/off a fan or a heater, or adjust a heat or cool setting of a thermostat.

At a step 808, the animal monitoring and communication device 310 may receive an action indicator. The action indicator may be an action to be taken in the environment (e.g., enclosure 12). For example, the action indicator may be an indication to modify a parameter of the environment device 306, such as turning the device on/off or modifying a setting (e.g., increase, decrease, switch modes, and so on) of the device. In some instances, the user device 302 transmits the action indicator, such that a user indicates the action to be taken. In some implementations, the action indicator may be an indication to take an action on the wearable device.

At a step 808, the animal monitoring and communication device 310 initiates an action based on the action indicator. The action may be taken in the environment of the animal enclosure by adjusting a parameter of an environment device. For example, the action may actuate a fan, a heater, a light, cause a sound to be generated, cause an environment device to be turned on and/or off, modify an intensity of an environment device, and so on, via a transmission from the animal monitoring and communication device 310 based on the action indicator. The action may be an action of a wearable device. For example, the wearable device may vibrate, make a sound, and so on, based on the action indicator. In some cases, the alert may be updated and/or the action may cease upon the animal condition and/or environment condition to a baseline level, or crossing the threshold in the opposite direction. For example, the environment device may be returned to a state before the action was taken. In some instances, an updated alert may be transmitted to the user device 302 indicating the first condition has crossed the threshold toward a baseline.

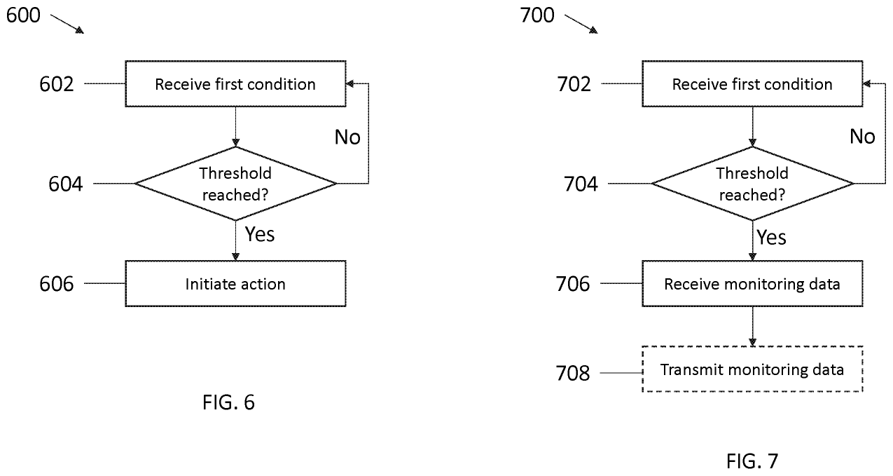

Referring to FIG. 9, a method 900 is provided for initiating an action based on a received threshold value with the animal monitoring and communication device 310. The method may be implemented using the communication systems of FIGS. 5A-5C.

At a step 902, the animal monitoring and communication device 310 may receive a threshold value. The threshold value may be preset and received from a databased based on the condition or conditions being monitored. The threshold value may be received from the user device 302, such that the user may set the threshold value. The threshold may be received from a machine learning system and/or an artificial intelligence system that modifies the threshold based on monitoring data. The machine learning system and/or an artificial intelligence system may modify and provide the threshold based on monitoring data for a first condition or monitoring data including multiple conditions. The machine learning system and/or an artificial intelligence system may modify and provide the threshold based on specific characteristics of the target animal 14.

At a step 904, the animal monitoring and communication device 310 may receive a first condition. Receiving the first condition at step 904 may be substantially similar to receiving a first condition at step 602 of method 600.

At a step 906, the animal monitoring and communication device 310 may compare the first condition to a threshold. Comparing the first condition to a threshold at 906 may be substantially similar to comparing the first condition to a threshold at step 604 of method 600. If the threshold is not reached, the method 900 returns to step 904, receiving the first condition.

If the threshold is reached, at a step 908, the animal monitoring and communication device 310 initiates an action. Initiating an action at 908 may be substantially similar to initiating an action at step 606 of method 600. Initiating an action may include initiating multiple actions. Initiating an action may include modifying the threshold value based on the threshold being reached for the first condition. In some instances, the animal monitoring and communication device 310, or a machine learning system and/or an artificial intelligence system, monitors a frequency at which the threshold is reached, and modifies the threshold based on the frequency of occurrence.

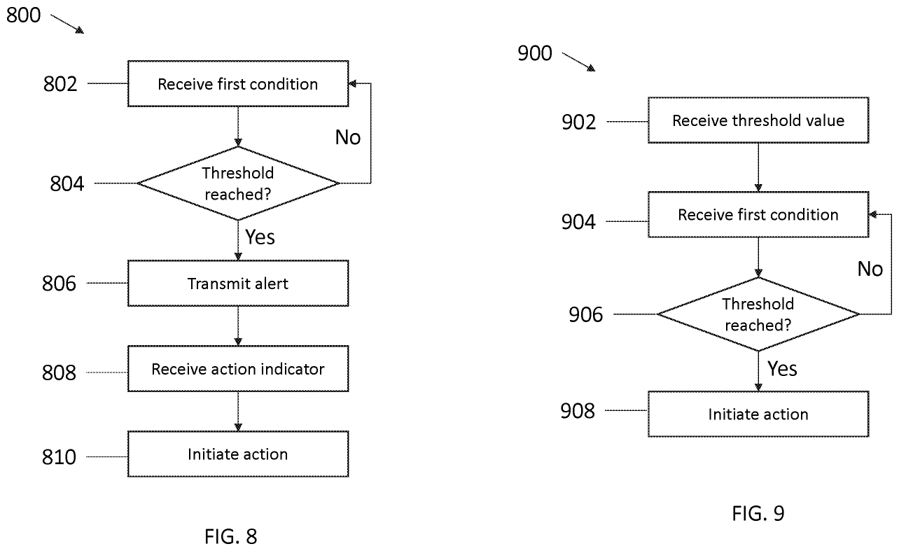

Referring to FIG. 10, a method 1000 is provided for initiating an action based on two conditions reaching a threshold with the animal monitoring and communication device 310. The method may be implemented using the communication systems of FIGS. 5A-5C. At a step 1002, the animal monitoring and communication device 310 may receive a first condition. Receiving the first condition at step 1002 may be substantially similar to receiving a first condition at step 602 of method 600. In some instances, the first condition is an environment condition. For example, the first condition may be a temperature, a humidity, a light level, a movement of the target animal 14, a sound, or a pressure.

At a step 1004, the animal monitoring and communication device 310 may compare the first condition to a first threshold. Comparing the first condition to a first threshold at 1004 may be substantially similar to comparing the first condition to a threshold at step 604 of method 600. If the threshold is not reached, the method 1000 returns to step 1002, receiving the first condition.

If the threshold is reached, at a step 1006, the animal monitoring and communication device 310 may receive a second condition monitoring data. For example, the second condition may be received from sensor(s) 304. The second condition may be an environmental condition of the animal enclosure 14. For example, the second condition may be an ambient temperature, a humidity, a light level, a movement of the target animal 14, a sound, or a pressure. The second condition may be an animal condition. For example, the second condition may be a temperature, heart rate, respiration rate, blood glucose level, or oxygen level of the target animal 14. The second condition may be one or more conditions. The first condition crossing the threshold may trigger collection and storage of the second condition monitoring data.

At a step 1008, the animal monitoring and communication device 310 compares the second condition to a second threshold. The second threshold may be a threshold related to the second condition. The second threshold may be a predefined threshold. For instance, the second threshold may be a threshold of the environmental condition. The second threshold may be a threshold of the animal condition. The second threshold may be predefined by a user. In some instances, when the second condition is more than one condition, if a single condition meets their respective threshold, the second threshold of step 1008 is reached. In another instance, when the second condition is more than one condition, a designated number of conditions reach meet their respective thresholds for the threshold of step 1008 to be reached. If another instance, when the second condition is more than one condition, all conditions must reach their respective thresholds for the second threshold of step 1008 to be reached. In some instances, the second threshold may only be met if the first threshold of step 1004 has been reached met. If the threshold is not reached, the method 1000 returns to step 1006, receiving the second condition.

If the threshold is reached, at a step 1010, the animal monitoring and communication device 310 initiates an action. Initiating an action at 1010 may be substantially similar to initiating an action at step 606 of method 600.

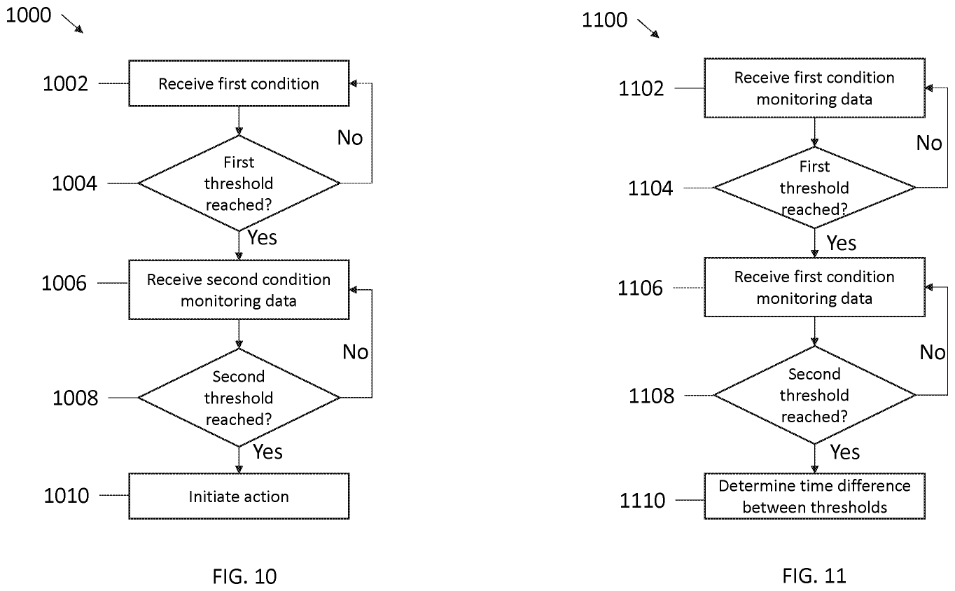

Referring to FIG. 11, a method 1100 is provided for monitoring a recovery of an animal or an environment return with the animal monitoring and communication device 310. The method may be implemented using the communication systems of FIGS. 5A-5C.

At a step 1102, the animal monitoring and communication device 310 may receive first condition monitoring data. The first condition monitoring data may be received when the target animal 14 returns to the enclosure 12, or is proximal the enclosure 12. For example, a wearable device may record the first condition monitoring data, and transmit the first condition monitoring data upon the wearable device entering a predefined radius of the animal monitoring and communication device 310.

At a step 1104, the animal monitoring and communication device 310 may compare the first condition to a first threshold. Comparing the first condition to a first threshold at 1104 may be substantially similar to comparing the first condition to a threshold at step 604 of method 600. In some instances, only a most recent subset of data is compared to the threshold. Upon the first threshold being reached by the first condition, the animal monitoring and communication device 310 may store a timestamp, time indicator, or start a timer. If the threshold is not reached, the method 1100 returns to step 1102, and receive first condition monitoring data when the animal monitoring and communication device 310 identifies a wearable device has returned to a radius of the animal monitoring and communication device 310.

If the threshold is reached, at a step 1106, the animal monitoring and communication device 310 may receive first condition monitoring data in real time. The first condition monitoring data is the same condition as the first condition of step 1102. For example, animal monitoring and communication device 310 may receive the real time data from the wearable device. In some instances, the animal monitoring and communication device 310 may receive the same type of condition data from a different sensor. In some instances, when the first threshold is reached, the animal monitoring and communication device 310 may subsequently initiate an action, such as an alert or modifying a parameter of an environment device 306 to modify the environment of the enclosure 12 and target animal 14.

At a step 1108, the animal monitoring and communication device 310 may compare the first condition to a second threshold. Comparing the first condition to the second threshold at 1108 may be substantially similar to comparing the first condition to a threshold at step 604 of method 600. The second threshold may be different from the first threshold. Upon the second threshold being reached by the first condition real time data, the animal monitoring and communication device 310 may store a timestamp, time indicator, or end a timer. If the threshold is not reached, the method 1100 returns to step 1106, and continues to receive first condition monitoring data in real time.

If the threshold is reached, at a step 1110, the animal monitoring and communication device 310 may determine a time difference between the first threshold being reached and the second threshold being reached. The time difference may indicate an amount of time it took for a condition of the animal to recover to a baseline (e.g., the second threshold). The time difference may indicate an amount of time it took for a condition of the environment to return to a baseline (e.g., the second threshold). In some instances, the amount of time may be used to modify a threshold. In some instances, the amount of time may be used to modify actions related to parameters of environment devices (e.g., adjust a level or intensity of an environment device).

Referring to FIGS. 6-11 generally, in cases where monitoring is activated prior to issuing one or more alerts, the animal monitoring and communication device 310 may continue monitoring animal behavior and/or environmental conditions to determine whether the threshold behavior and/or condition continues, e.g., for a predefined time, up to a predefined number, for a time and/or number established by the user, by machine learning models, or by use of artificial intelligence, or artificial intelligence agent. For example, artificial intelligence, or artificial intelligence agents may be used for timing of devices, automation, or a combination thereof. The artificial intelligence or artificial intelligence agents may be used to analyze datasets, identify trends or patterns, and autonomously modify thresholds, environment parameters, or a combination thereof.

Alerts may be issued by the animal monitoring and communication device 310 or the device may cause alerts to be issued, e.g., by the docking station 20 or a communicatively coupled device. The alerts may be received on a user device, e.g., mobile phone, communicatively coupled to the animal monitoring and communication device remote device, e.g., via a network.

Implementations of the present disclosure may be useful in animal shelter organizations that regularly use animal crates to care for and transport animals in need, and for instance the animal monitoring and communication system 10 may facilitate tracking and caring for the animal while in transit to keep the animal calm and safe. Veterinary clinics may use the animal monitoring and communication system 10 in connection with confined spaces, e.g., kennels, to allow an owner to care and soothe the pet while their pet remote and staying away from home. Alternatively, veterinary clinics may remotely monitor an animal's health from the animal's home, saving money for the owner. Animals with anxiety may find comfort in using a confined space configured with the animal monitoring and communication system 10. In the case of natural disasters, the animal monitoring and communication device may be used in connection with animal crates to assist with reuniting pets and owners. In examples where the animal is remotely located from their typical residence, the animal monitoring and communication system 10 may include identity chip readers, which may be used to verify the animal's presence in the confined space. Police enforcement and related industries may use the animal monitoring and communication system 10 in connection with a K9 in a vehicle. In that instance, the animal monitoring and communication system 10 may be communicatively coupled to operations within the vehicle, such as the air conditioning, heat, the windows, the lights, and so on, to modify the environment of the animal by modifying settings of the vehicle.

What is claimed is:

1. An animal monitoring and communication system, comprising:
a first sensor configured to be positioned proximal to an environment of an animal;
an environment device configured to modify the environment of the animal; and
a processor communicatively coupled to the first sensor and to the environment device, wherein the processor is configured to:
receive a first condition from the first sensor;
compare the first condition to a first threshold condition; and
initiate an action within the environment of the animal in response to the first condition reaching the first threshold condition.

2. The system of claim 1, wherein the first sensor includes one or more of a camera, thermometer, a humidity sensor, a light sensor, or a combination thereof.

3. The system of claim 2, wherein the initiated action causes the processor to receive monitoring data from the first sensor.

4. The system of claim 2, wherein the processor is communicatively coupled to a communications network and configured to transmit monitoring information to a communications hub.

5. The system of claim 2, wherein the processor is communicatively coupled to a user interface and configured to transmit monitoring information thereto.

6. The system of claim 5, wherein the processor is configured to receive input from the user interface for controlling the environment device.

7. The system of claim 6, further comprising a user device communicatively coupled to the processor, the user device configured to display the user interface,
wherein the user device is configured to be docked in a docking station of the animal monitoring and communication system.

8. The system of claim 7, wherein the processor is configured to transmit monitoring information to the user device upon being docked in the docking station.

9. The system of claim 8, wherein the docking station comprises a housing configured to be attached to a housing of the environment of the animal,
wherein the housing of the environment of the animal is an animal crate or an animal stall.

10. The system of claim 1, wherein the processor, the first sensor, and the environment device are integrated into a single assembly.

11. The system of claim 1, wherein the processor, the first sensor, and the environment device are arranged in the environment of the animal and remotely located from each other.

12. The system of claim 1, further comprising a second sensor configured to monitor a second condition, wherein the second sensor and the processor are arranged in the environment of the animal and remotely located from each other.

13. The system of claim 12, wherein the second sensor includes one or more of a clothing article, a collar, a saddle blanket, a mat, an implantable chip, or a combination thereof, or wherein the second condition is one or more of an animal temperature, a heart rate, a respiration rate, a blood glucose level, an oxygen level, or a combination thereof, or
wherein the initiated action causes the processor to receive monitoring data from the second sensor.

14. The system of claim 1, wherein the first threshold condition is predefined by a user.

15. The system of claim 1, wherein the first threshold condition is generated by one or more of a machine learning model, artificial intelligence, or a combination thereof.

16. The system of claim 1, wherein the environment device includes one or more of a fan, a light, a speaker, a feeding device, an electric device, or a combination thereof.

17. The system of claim 1, wherein the initiated action causes the environment device to modify the environment of the animal.

18. A system comprising:
one or more hardware processors; and
a memory storing instructions that, when executed by at least one of the one or more hardware processors, causes the system to perform operations comprising:
receiving a first condition from a first sensor proximal to an environment of an animal;
comparing the first condition to a first threshold condition; and
initiating a first action within the environment of the animal in response to the first condition reaching the first threshold condition.

19. The system of claim 18, wherein the operations further comprise:
receiving a second condition from a second sensor;
comparing the second condition to a second threshold condition; and
initiating a second action within the environment of the animal in response to the second condition reaching the second threshold condition.

20. A non-transitory computer-readable storage medium comprising instructions that, when executed by one or more hardware processors of a system, cause the system to perform operations comprising:
receiving a first condition from a first sensor proximal to an environment of an animal;
comparing the first condition to a first threshold condition; and
initiating a first action within the environment of the animal in response to the first condition reaching the first threshold condition.
